(19) United States
(12) Patent Application Publication
(10) Pub. No.: US 2012/0077860 A1
(43) Pub. Date: Mar. 29, 2012

(54) Adeno-Associated Viral Vector for Exon Skipping in a Gene Encoding a Dispensable Domain Protein

(75) Inventor: Luis Garcia, Saint Denis (FR)

(73) Assignees: GENETHON, Evry (FR); CENTRE NATIONAL DE LA RECHERCHE SCIENTIFIQUE, Paris (FR)

(21) Appl. No.: 11/573,811

(22) PCT Filed: Aug. 2, 2005
(86) PCT No.: PCT/FR05/50642

§ 371 Date: Feb. 16, 2007

(30) Foreign Application Priority Data
Aug. 17, 2004 (FR) 0451861

(51) Int. Cl. A61K 31/7052; C12N 5/06; C12N 15/00; A01K 67/033

(52) U.S. Cl. 514/44 A; 435/1.1; 435/320.1; 435/325; 800/13

(57) ABSTRACT

The invention concerns an adeno-associated viral vector comprising:
a U7 type modified snRNA sequence;
the native U7 promoter;
at least one antisense sequence directed against at least one splice site of at least one exon, the said exon encoding a dispensable domain of dystrophin.

This invention relates to the use of adeno-associated viral vectors, or AAV vectors, to deliver antisense sequences, directed against splice sites of a gene encoding a dispensable domain protein, to target cells, and the therapeutic applications of this, in particular in the treatment of Duchenne muscular dystrophy.

Thus, carefully chosen sequences introduced into a vector according to the invention are capable of resulting in transcripts producing a shorter but functional dystrophin protein, correcting certain forms of Duchenne muscular dystrophy.

Duchenne muscular dystrophy (DMD) is a genetic disease carried on the X chromosome, which affects about 1 boy in 3,500. It is characterised by the absence of a 427 kilodalton protein, cytoskeletal dystrophin, which results in the death of muscle fibres, correlated with progressive severe muscle deterioration.

Dystrophin is a modular protein with a central region composed of 24 spectrin-like repeated domains. Proteins lacking certain of these repeated sequences can however be perfectly functional or at least only partially defective, as observed in attenuated forms of DMD (Becker dystrophy).

On the other hand, the majority of serious mutations of the dystrophin gene consist of deletions of one or more exons perturbing the final messenger's reading frame or point mutations, present in the encoding regions or exons, which introduce stop codons or shift the reading phase. In both cases, these mutations result in the absence of dystrophin.

As an illustration, a large number of clinical cases of Duchenne muscular dystrophy are linked to multi-exon deletions (severe DMD genotypes: Δ45-50; Δ47-50; Δ48-50; Δ49-50; Δ50; Δ52) where the reading frame could be re-established by deleting exon 51 (mild BMD genotypes: Δ45-51; Δ47-51; Δ48-51; Δ49-51; Δ51-52).

Different strategies and techniques have been envisaged to attempt to “repair” the mutated dystrophin genes.

Replacing the dystrophin gene in affected muscle fibres, or compensating for necrosed cells by healthy cell grafts, has revealed major difficulties.

The third route envisaged, currently the most used, consists of attempting to repair the mutated DNA using antisense oligonucleotides (or AON) allowing certain exons to be skipped, thus to arrive at the expression of a truncated but functionally effective protein. This so called “exon skipping” technique involves the use of oligonucleotides which are complementary to the sequences involved in splicing the exon to be masked.

Current studies in this direction are essentially being carried out in mdx mice which have this disease, because of a nonsense mutation introducing a stop codon in exon 23 of the murine dystrophin gene.

The main difficulty of this technology lies in introducing a non-degraded oligonucleotide (AON) in a stable and sustainable manner into diseased muscle fibres, particularly in vivo.

In the first instance, directly injecting the said oligonucleotides was envisaged, possibly in combination with a synthetic detergent, such as the agent F127. Advantageous sequences to be administered by this route to mask exon 19 or 45 of the human dystrophin gene are for example thus described in patent applications EP 1054058 and EP 1191098, respectively. However, in view of the short life span of these oligonucleotides in muscle, this method of treatment requires regular, relatively restricting injections.

In addition, attempts have been made to introduce these sequences into vectors to carry them into the target cells. To date, only in vitro trials have been carried out: the constructions used are based on the use of retroviruses and have been tested only on cell cultures. The results reported have not proved to be very convincing, or at least insufficient to envisage an in vivo transposition.

For example, the document WO 02/24906 illustrates the use of these two distinct methods to exclude exon 46 in human cells.

All of the work carried out on vector transfer of AON has mainly shown that:

The presence of small snRNA (small nuclear RNA) type sequences allows better translocation into the nucleus of target cells and better transcription of sequences. The document WO 03/095647 thus advocates the advantages of selecting U2 and U3 snRNA.
i) The simultaneous presence of two target sequences involved in splicing the same exon improves the efficiency of skipping that exon (1, 2).

However, at present no solution has been proposed for skipping exons in vivo, with the exception of injecting oligonucleotides with the disadvantages indicated above.

For the first time, the inventors propose a construction producing in vivo results which are remarkable as far as restoring dystrophin protein activity is concerned in Duchenne muscular dystrophy.

In addition, these results obtained on dystrophin could be extended to any dispensable domain protein. It could also concern all multi-exon genes encoding a dispensable domain protein where the deletion (by exon skipping) does not affect or little affects the activity of the protein.

In regard to this, the invention proposes to construct an adeno-associated viral vector comprising:
a modified U7 snRNA sequence;
the native U7 promoter;
at least one antisense sequence directed against at least one splice site of at least one exon, the said exon encoding a dispensable domain of dystrophin.

In the rest of the description, the combination composed of these three entities (an snRNA sequence/U7 promoter/antisense sequence or sequences) is called the “U7 cassette”.

Among the multitude of available vectors, the applicant has selected to advantage a vector of viral origin, namely an adeno-associated virus derivative or AAV. Among the 8 identified serotypes, the AAV used in the particular context of DMD is preferably an AAV1, i.e. it has a serotype 1 capsid. Indeed, AAV1 most effectively transduces muscle cells.

On the other hand, the original viral sequences, in particular ITRs associated with the transgene are to advantage from AAV2. The result is that, in an advantageous embodiment, the final adeno-associated viral vector is a 2/1 pseudotype.

The said vector also contains a modified snRNA sequence. Small nuclear RNAs, or snRNAs, are RNAs of small size present in the nucleus of cells and implicated in certain stages of maturation of pre-mRNAs. They are called U1, U2 . . . U10.

Among these different types of snRNA, type U7, normally involved in the maturation of premessenger RNAs encoding histones, is preferentially used as transporter.

The snRNAs in question may be of human or murine origin in as far as these small sequences are highly conserved between the different species. Preferentially, the snRNA used in the invention is that of the mouse.

“Modified snRNA” means an RNA in which the sequences involved in the initial function of the snRNA are inactivated. These sequences may also be modified in such a way as to increase the level of expression of the said snRNA.

For example, in the case of U7, the sequence of the fixation site of the “small nuclear ribonucleoprotein” (or Sm protein) is modified so as to inactivate the maturation of premessenger RNAs encoding the histones and to increase in parallel the nuclear concentration of U7snRNA. Moreover, the sequence of 18 complementary nucleotides at the 3′ maturation site of premessenger RNAs encoding the histones is replaced by the antisense sequences of interest.

In practice, these modifications can be introduced by site-directed mutagenesis using PCR.

The snRNA gene thus modified is then cloned in the AAV vector, preferably between its two ITR sequences.

This invention may also be carried out with U1 or U2 sequences, but with more modifications and a less effective result.

A vector according to the invention also includes a promoter allowing antisense sequences to be expressed at a sufficient level to ensure their biological and therapeutic activity. Numerous usable promoters in the context of AAVs are known to those skilled in the art. However, in a preferred embodiment of the invention, the expression of the antisense sequences is controlled by the native promoter of the snRNA used in the construction. Where U7 is preferred, it is thus the U7 promoter which transcribes the antisense sequences.

The vector according to the invention also includes at least one antisense sequence directed against at least one splice site of at least one exon (i.e. capable of interfering with the splicing of the said exon). The antisense sequence is preferentially a complementary sequence with at least one sequence chosen from the following group: 5′ splice site (donor); 3′ splice site (acceptor); BP (Branching Point) intronic sequence; and possibly purine-rich internal regions, more specifically ESEs (Exon-internal Splicing Enhancers).

Advantageously, to ensure the exclusion of a given exon, two antisense sequences with distinct targets, preferentially the 5′ donor site and the BP sequence, are introduced in a single recombinant vector according to the invention.

Antisense sequences directed against splice sites of at least two distinct exons may also be associated in the same construction.

Alternately, it is possible to use several constructions, each carrying a distinct antisense sequence, the said sequences being directed against one or more exons.

In practice, when several antisense sequences are combined (directed against the same exon or several different exons), the following situations can arise:
the antisense sequences are integrated into the same U7 cassette, carried by a single AAV vector; or
the antisense sequences are integrated into different U7 cassettes, carried by a single AAV vector; or
the antisense sequences are integrated into different U7 cassettes, each carried by an AAV vector.

In the context of the invention, the antisense sequences are specific to the different splice sites of exons forming the dystrophin gene, whatever its origin.

The murine dystrophin gene is of obvious interest as the mouse is an experimental animal model of choice. Thus, an mdx mouse, with a mutation in exon 23 of the murine dystrophin gene producing an inactive truncated protein, shows the symptoms of DMD. In this context, the antisense sequences are directed therefore against the sequences involved in splicing exon 23.

More particularly, a vector according to the invention includes an SEQ ID 1 sequence consisting of a U7snRNA gene modified as described above and integrating antisense sequences directed against the 5′ donor site (SEQ ID 2) and the BP sequence (SEQ ID 3) of exon 23 of the murine dystrophin gene placed under the control of the U7 promoter, introduced between the 2 ITR sequences of the AAV vector.

Very interestingly, the functionality of such vectors has been validated by the applicant in a large animal, the dog. There are indeed dogs that are naturally myopathic because of a mutation in the splice acceptor site of intron 6 such that the fact that exon 7 is not taken into account in the final mRNA nullifies the reading frame, exons 6 and 8 not being in phase. Theoretically an operational reading frame can be restored by simultaneously skipping exons 6 and 8, the final mRNA being thus deprived of exons 6, 7 and 8. The association of vectors according to the invention, including the antisense sequences SEQ ID 27 and 28, directed against ESE regions of exons 6 and 8 respectively, has thus proved effective.

For treatment in humans, the antisense sequences selected are directed against at least one splice site of at least one exon of the human dystrophin gene, the exclusion of which produces a truncated but active protein.

As previously discussed, exon 51 of the human dystrophin gene, and more particularly the sequences involved in its splicing, are advantageous targets in the context of the invention. Thus, excluding it from the transcript encoding dystrophin may be beneficial in the treatment of about 20% of the clinical cases today recorded as genotyped for Duchenne muscular dystrophy.

It has been shown by the inventors that a suitable construction of the invention comprised the sequence SEQ ID 4, associating two antisense sequences SEQ ID 5 and SEQ ID 6 directed against purine-rich internal regions of exon 51 of the human dystrophin gene, in place of sequences SEQ ID 2 and 3 in the sequence SEQ ID 1.

Other human antisense sequences that can be used in the context of the invention are as follows:
DA5′ of sequence SEQ ID 7 directed against the 5′ site of exon 51;
DA3′ of sequence SEQ ID 8 directed against the 3′ site of exon 51;
G5′ of sequence SEQ ID 9 directed against the 5′ site of exon 51;
GBP of sequence SEQ ID 10 directed against the BP site of exon 51;
ESE4, ESE16 and ESE28 of sequence SEQ ID 11, 12 and 13, respectively, directed against ESE type sites of exon 51;
Ex51AONlong1 of sequence SEQ ID 25;
Ex51AONlong2 of sequence SEQ ID 26.

These sequences are preferentially combined in tandem in a vector according to the invention. Preferentially, DA5′ (SEQ ID 7) and DA3′ (SEQ ID 8) or G5′ (SEQ ID 9) and GBP (SEQ ID 10) are combined.

Sequences SEQ ID 25 and SEQ ID 26 correspond to the longer sequences SEQ ID 5 and SEQ ID 6. They are preferentially combined. Given their large size, each is advantageously integrated in a U7 cassette, carried either by the same AAV vector, or by two distinct AAV vectors used in tandem.

Very interestingly, the applicant has shown in this invention that the nature of the antisense sequence could play an important role in the efficacy of the construction and also that it was possible to evaluate the efficacy of these sequences by in vitro experiments on target muscle cells by transposing the said U7 cassettes in a lentiviral vector.

The invention thus also concerns a lentiviral vector comprising:
a U7 type modified snRNA sequence;
the native U7 promoter;
at least one antisense sequence chosen from the group consisting of SEQ ID 2, SEQ ID 3, SEQ ID 27, SEQ ID 28, SEQ ID 5, SEQ ID 6, SEQ ID 7, SEQ ID 8, SEQ ID 9, SEQ ID 10, SEQ ID 11, SEQ ID 12, SEQ ID 13, SEQ ID 25, SEQ ID 26.

The applicant has shown that such a vector is effective in skipping the human exon 51, considerably more so than seen with the construction of De Angelis et al. (1). For the first time, the unequivocal production of dystrophin in vivo is reported, after the injection of patient cells (delta 49-50) genetically modified using a lentiviral vector according to the invention into SCID-mdx mice.

Preferentially, the lentiviral vector replacing the adeno-associated viral vector according to the invention is an SIN (self inactivating) lentivirus of the latest generation. Nevertheless, any lentiviral vector may to be used to insert a U7 cassette. The construction and manipulation of lentiviral vectors is well known to those skilled in the art.

These lentiviral vectors have applications which are complementary to those of recombinant AAVs according to the invention. While the latter are intended to be injected in situ in the target muscles, with lentiviral vectors the efficacy of potential antisense sequences can be tested in vitro, directly in differentiated muscle cells from the patient to be treated. Secondly, if these vectors are introduced into myoblasts, muscle precursors, or even stem cells, in situ grafting of transfected cells can be envisaged.

Any type of cell transfected by the recombinant vector is therefore also part of the invention, particularly muscle cells and in particular muscle fibre types (myotubes), muscular precursors (myoblasts) or any cell capable of muscular differentiation.

Any muscular tissue or non-human organism transfected by the said vector is also included within the scope of the protection sought. Among non-human organisms, animals—in particular mice—are preferred.

This application describes for the first time a therapeutic potential for the vectors claimed. This invention thus also concerns pharmaceutical compositions including at least one vector as defined in this application as an active component, as well as the use of this vector as a medicinal product. In addition, as indicated above with reference to lentiviral vectors, transfected cells may also have therapeutic potential in the context of grafts.

A pharmaceutical composition according to the invention contains the vector or the cells claimed, associated with a pharmaceutically acceptable, inert vehicle.

When vectors according to the invention are to be injected into diseased muscles, the pharmaceutical composition will preferably be in liquid form. The vector concentration, the quantity to be injected and the frequency of injections are easily determined by those skilled in the art.

In view of the remarkable effects seen in vivo on restoring dystrophin in muscle fibres affected by DMD, a claim is also made for using the vector or cells according to the invention to prepare a medicinal product intended for the treatment of Duchenne muscular dystrophy.

More generally, a vector according to the invention may be used for the treatment of any disease associated with dysfunction of a dispensable domain protein where skipping at least one exon encoding a dispensable domain can restore its function.

Apart from the dysfunction of dystrophin associated with Duchenne muscular dystrophy illustrated here, certain myopathies are linked for example to dysfunction of the dispensable domain protein dysferlin and could therefore be treated with a vector according to the invention.

During in vivo use, vectors according to the invention proved to be stable, to have a specific sub-cellular location and permanently produce therapeutically active quantities of antisense.

More generally, this application demonstrates the potential of the AAV-U7snRNA system as a tool for inactivating or modifying mRNA in animals.

The invention and the advantages resulting from it are better illustrated by the following embodiments and the attached figures.

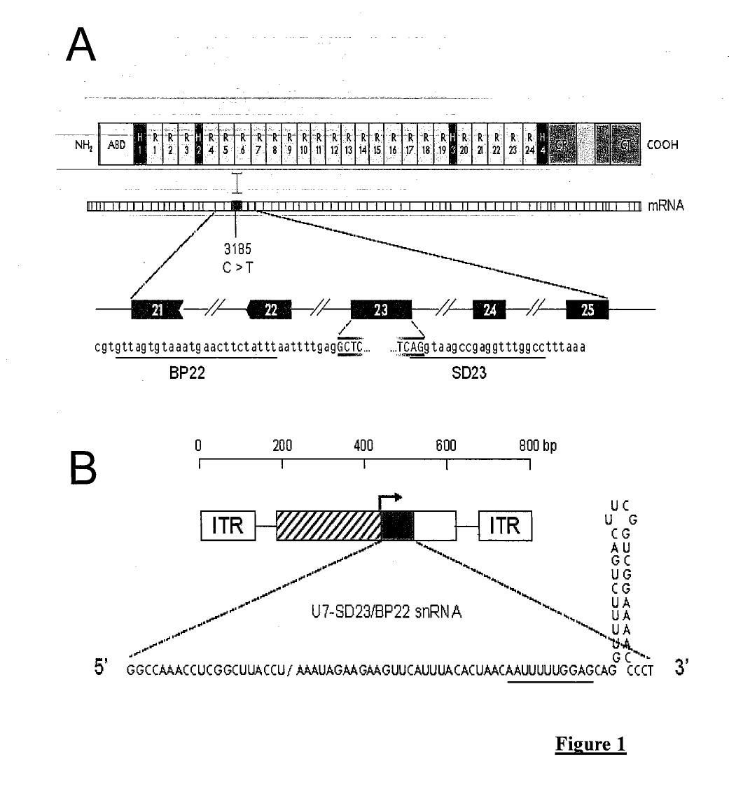

FIG. 1:
(A) The upper diagram schematically illustrates the structure of intact dystrophin (427 kDa). It consists of several domains: an N-terminal actin binding domain (ABD), a central rod domain composed of 24 spectrin-like repeats (R) and 4 hinge segments (H) capable of providing flexibility, and a cysteine rich domain (CR) which fixes β-dystroglycan and other members of the complex associated with dystrophin near the C-terminal end. The centre diagram represents dystrophin mRNA (about 14,000 bases) composed of 79 exons. In mdx mice, the replacement of a cytosine by a thymidine in exon 23 at position 3185 of the coding sequence creates a premature stop codon. The bottom diagram shows the target sequences at the BP sequence (branch point; BP22; SEQ ID 3) before exon 23 and after the splice donor site (SD23; SEQ ID 2) to force the mechanism controlling splicing to skip the mutated exon while keeping a reading frame open.
(B) Structure of the AAV(U7-SD23/BP22) vector. The U7-SD23/BP22 cassette is composed of the U7 promoter (position −267 to +1, hatched box), the modified U7 snRNA sequence (grey box and corresponding sequence below) and the sequences after position 116 (white box). This cassette has been placed between two inverse terminal repeats (ITR) of AAV2 (SEQ ID 1).

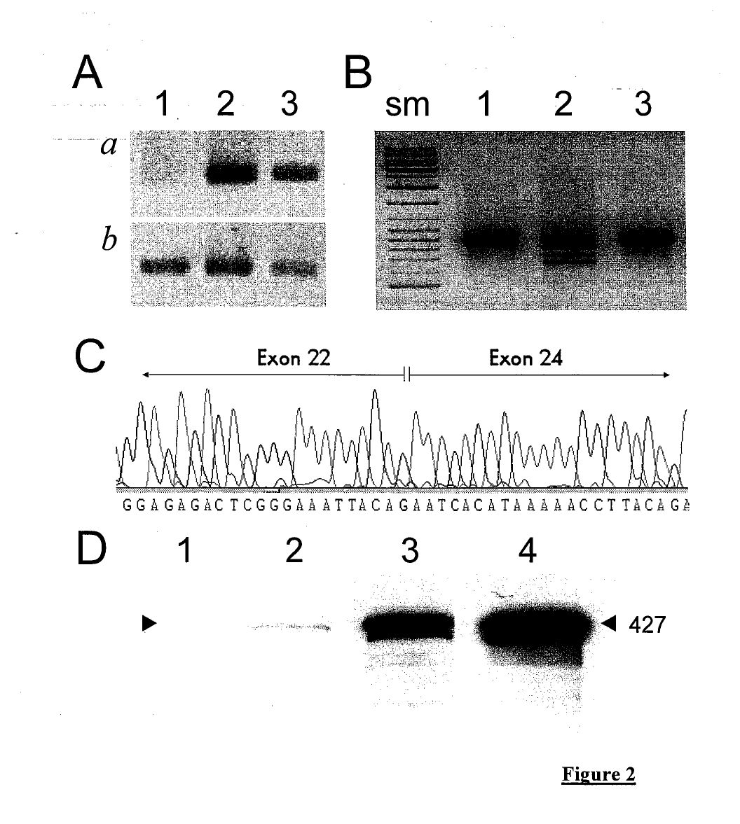

FIG. 2:
(A) Detection of the U7-SD23/BP22 modified snRNA; (a) and the endogenous U7 snRNA (B) following intramuscular injection of the AAV vector. Samples of total RNA were analysed at 0, 15 and 30 days (columns 1, 2 and 3, respectively) by RT-PCR. The products corresponding to 60 bp were visualised on agarose gel. Detection of the dystrophin mRNA with skipping of exon 23. Samples of total RNA were analysed at 0, 15 and 30 days by nested RT-PCR, using primer pairs in exon 20 and 26. The 901 by band which corresponds to the mRNA without the skip (*) is the only type detected on day 0 (column 1). It is gradually replaced by a 688 by fragment (**) which corresponds to the mRNA that has lost exon 23.
(B) DNA sequence of the 688 bp band after purification on gel.
(C) Immunodetection of total proteins extracted from tibialis anterior muscles, stained with anti-dystrophin Dys1 monoclonal antibodies (the arrows indicate the 427 kDa complete dystrophin: column 1, mdx, not injected; column 2, mdx, 2 weeks after injection; column 3, mdx, 1 month after injection; column 4, C57B16). Each column was loaded with a total quantity of protein of 40 μg. The same profile was seen with Dys2 antibodies (results not shown).

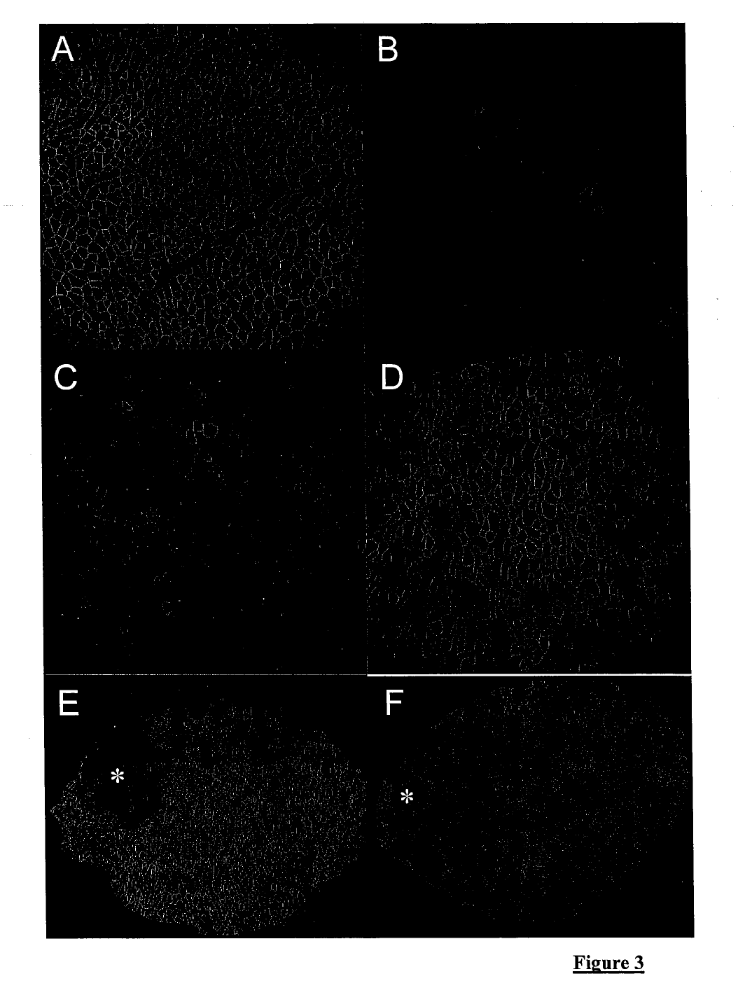

FIG. 3: Restoration of dystrophin in mdx mice after AAV (U7-SD23/BP22) administration. Immunological staining with Dys2-Ab of complete transverse sections of the hind limb anterior compartment (tibialis anterior muscle=TA and extensor digitorum longus*=EDL) of (A) normal C57B16, (B) untreated mdx, (C-E) mdx mice 2, 4 and 12 weeks after intramuscular injection, and (F) mdx 4 weeks after intra-arterial vector release. Scales (A-D): 0.5 mm; (E-F): 1 mm.

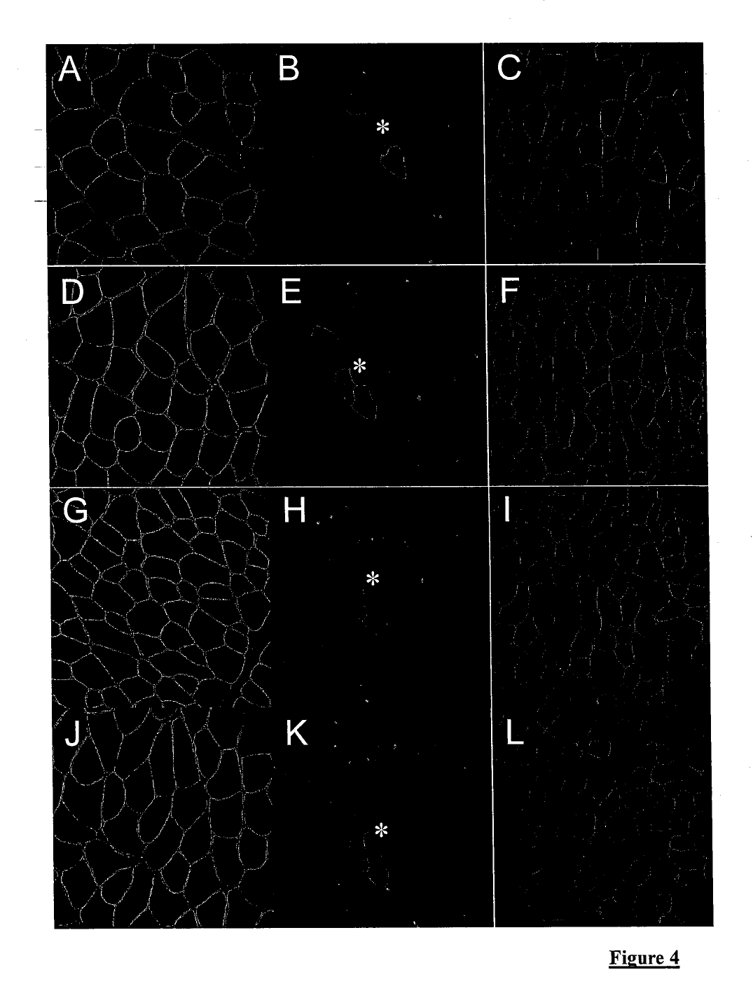

FIG. 4: Restoration of the protein complex associated with dystrophin in treated mdx muscles. The left, centre and right columns respectively show sections of the tibialis anterior muscle of normal C57B16, untreated mdx and mdx mice after treatment. The sections have been immunostained for dystrophin (A,B,C), for α-sarcoglycan (D,E,F), β-sarcoglycan (G,M,I) and β-dystroglycan (J,K,L). The same set of revertant fibres (*), showing dystrophin and the associated complex protein, is shown in the series of sections from untreated mice.

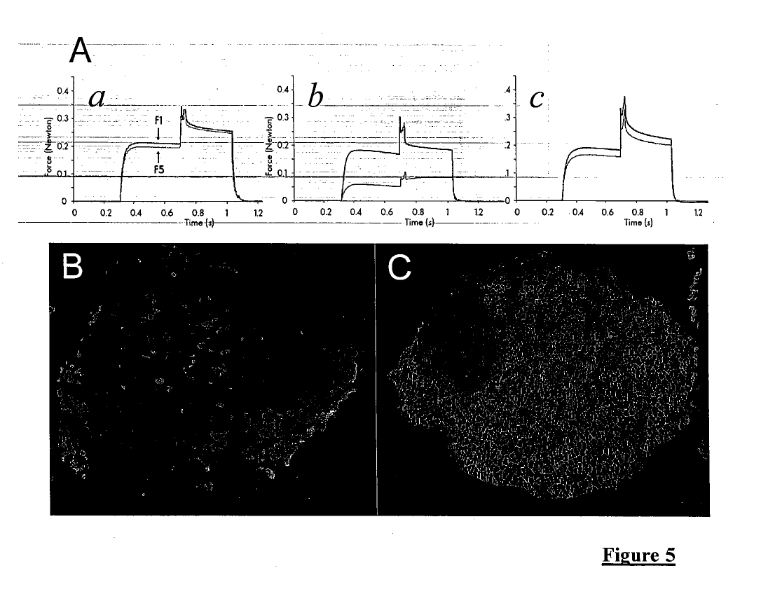

FIG. 5: The restoration of dystrophin in treated mdx muscles re-establishes normal susceptibility to damage induced by exercise. (A) Superimposed recordings of the tension produced by the EDL muscles of a) C57B16, b) untreated mdx and c) mdx mice after 45 days of treatment, during 5 tonic contractions with forced extension. The isolated muscles were subjected to repetitive stimulation (125 Hz) for 360 ms, at 3 minute intervals. During the first 160 ms, the tension developed isometrically, then an extension force corresponding to 10% of the length L₀, for which the muscle produced a maximum force, was imposed at a speed of 1 fibre length per second. After relaxation, the muscle returned to its resting length. The decrease in force was expressed as (F₁-F₅)/F₁, where F₁ is the isometric force developed just before extension in the first tetanic contraction, and F₅ that of the fifth. The decrease in force reached a mean of 15% for C57B16 muscles against 65% in mdx muscles. For the treated mdx shown in c), the decrease in force was reduced to 17%, indicating full reacquisition of the mechanical properties of the muscle fibres. (B and C). Detection, using Evans blue, of muscle fibres damaged by exercise in the tibialis anterior muscles of untreated (B) or treated (C) legs of the same mdx animal, 60 days after administration of the treatment. Damaged fibres incorporate Evans blue, the fluorescence of which is detected in the red channel. Dystrophin was immunologically stained with Ab-dys2 (green).

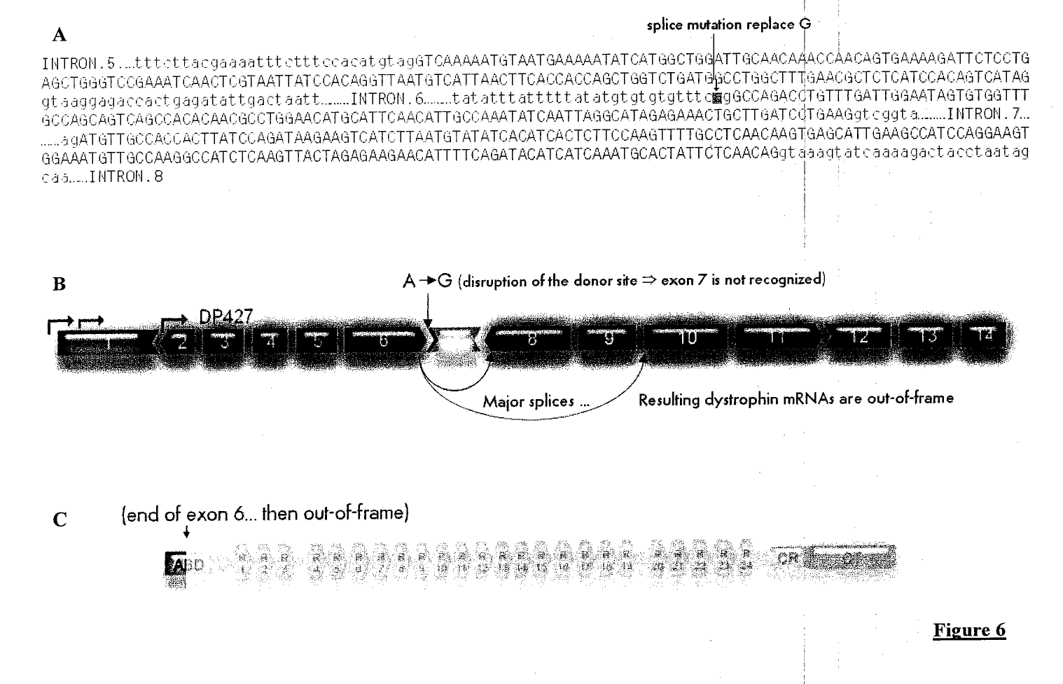

FIG. 6:
(A) Sequence of the intron 5-intron 8 region in the GRMD dog. The mutation responsible for the phenotype has been identified.
(B) Diagram of mRNA splicing in the GRMD dog.
(C) Diagram of the interruption of the reading phase at the end of exon 6 resulting in atrophied dystrophin.

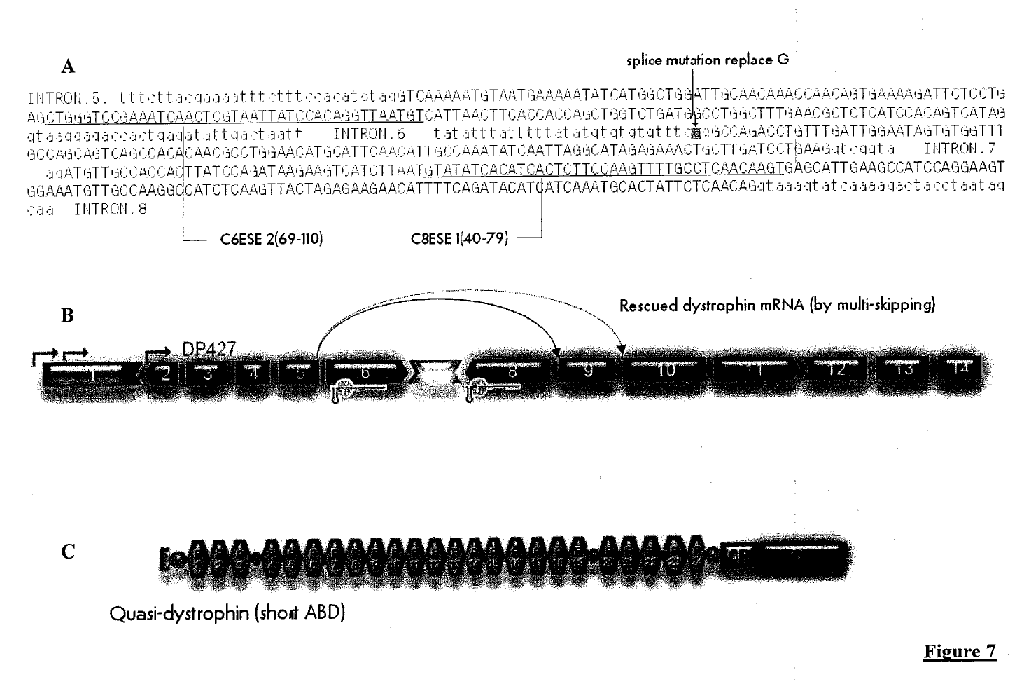

FIG. 7:
(A.) Location of target sequences situated in the ESE sequences of exon 6 (C6ESE2; SEQ ID 27) and exon 8 (C8ESE1; SEQ ID 28).
(B) Diagram of mRNA splicing in the GRMD dog, after multiple exon skipping carried out with antisense sequences C6ESE2 and C8ESE1.
(C) Diagram of the protein sequence of the dystrophin synthesized after multiple exon skipping.

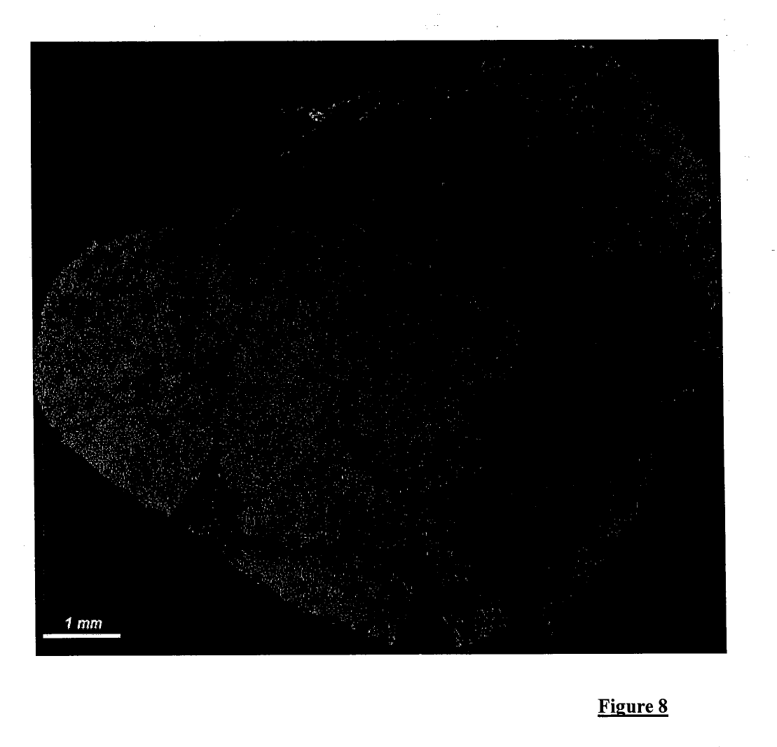

FIG. 8: Restoration of dystrophin in adult GRMD dogs, obtained 2 months after a single intramuscular injection of a preparation containing the AAV (U7-ex6) vector integrating the antisense sequence SEQ ID 27 and AAV (U7-ex8) vector integrating the antisense sequence SEQ ID 28. Immunological staining of complete transverse sections with Dys2-Ab. Scale 1 mm.

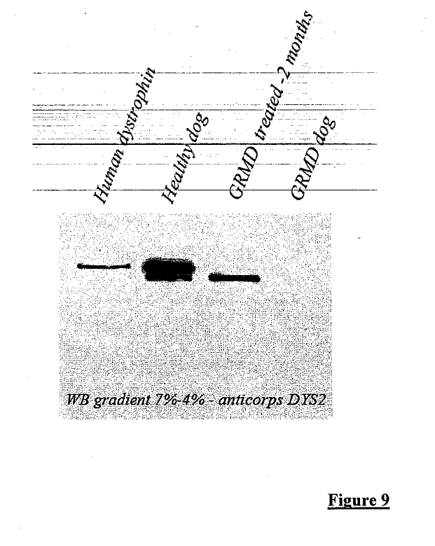

FIG. 9: Immunodetection of total proteins extracted from tibialis anterior muscles, stained with anti-dystrophin Dys2 monoclonal antibodies: column 1, human dystrophin in a healthy subject; column 2, dystrophin in a healthy dog, column 3, GRMD dog 2 months after treatment; column 4, untreated GRMD dog. Each column was loaded with a total quantity of protein of 40 μg.

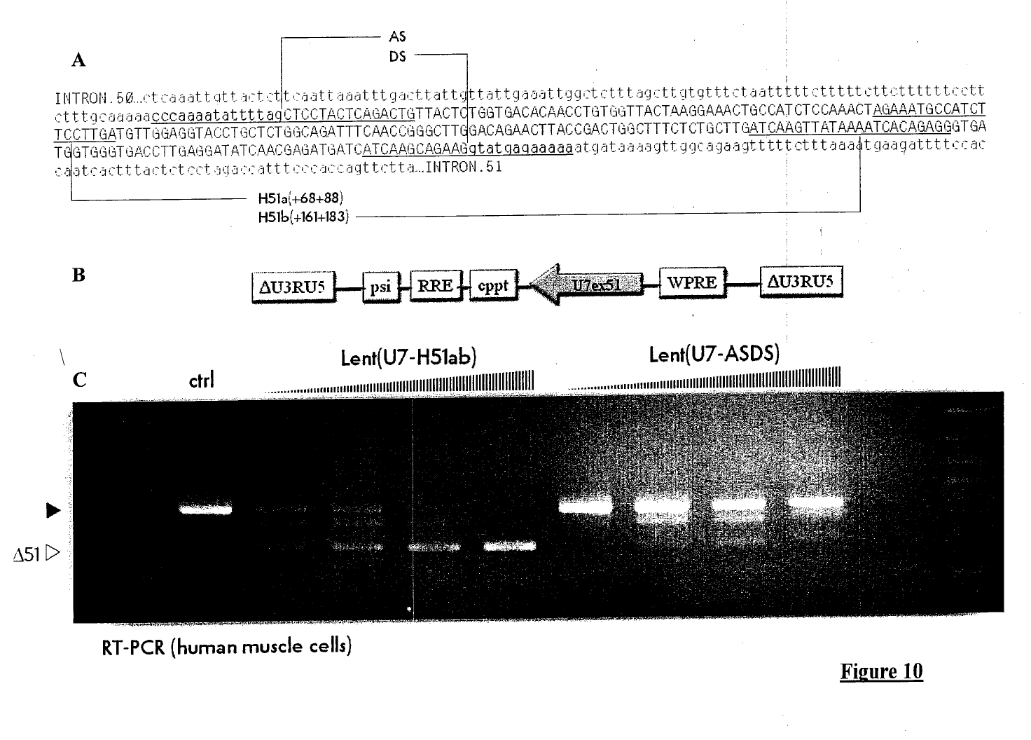

FIG. 10:
(A) Location of target sequences allowing skipping of exon 51 in the human dystrophin gene: the antisense sequence H51a has the sequence SEQ ID 6, and the antisense sequence H51b has the sequence SEQ ID 5. The antisense sequences AS and SD are as described by De Angelis (1).
(B) Diagram of the integration of the U7 cassette into the lentiviral vector.
(C) Detection of human dystrophin mRNA with skipping of exon 51. The samples of total RNA were analysed by nested RT-PCR. The black arrow corresponds to the mRNA without skipping (*), while the white arrow indicates the mRNA lacking exon 51.

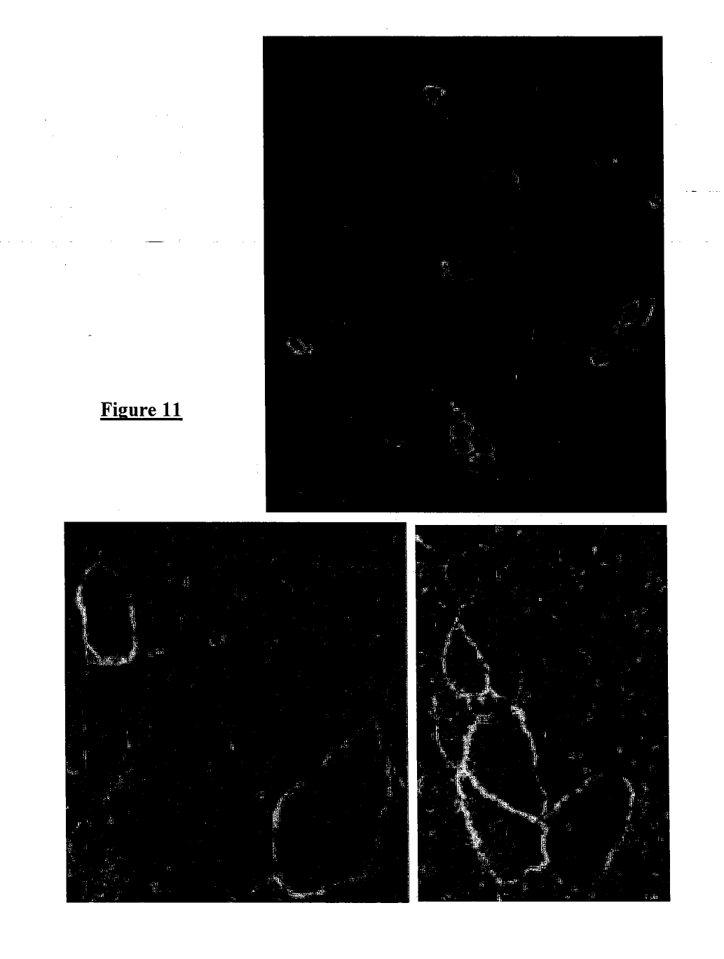

FIG. 11: Restoration of dystrophin in SCID-mdx mice, obtained one and a half months after injection into the tibialis anterior muscles of delta 49-50 stem cells transduced by the Lent (U7-H51ab) vector integrating the antisense sequences SEQ ID 5 and 6. Immunological staining with Dys3.

### A.—EXPERIMENTAL STUDY IN THE MOUSE

### I Material and Methods

1. Constructions

The entire U7 snRNA gene (445 bp) was obtained by PCR on the genomic DNA of the mouse with the oligonucleotides: 5′-TAACAACATAGGAGCTGTG-3′ (SEQ ID 14) and 5′-CAGATACGCGTTTCCTAGGA-3′ (SEQ ID 15). The Sm domain (AATTTGTCTAG; SEQ ID 16) was optimised to smOPT (AATTTTTGGAG; SEQ ID 17), as described previously (3), and the U7 region capable of matching with the pre-mRNA was exchanged with a sequence of 44 complementary nucleotides both in the region covering the BP (branch point) sequence before exon 23 of the dystrophin gene (BP22: 5′-AAATAGAAGTTCATTTACACTAAC-3′; SEQ ID 3) and the region after the splice donor site (SD23: 5′-GGCCAAACCTCGGCTTACCT-3′; SEQ ID 2). The resulting U7smOPT-SD23/BP22 fragment was then inserted between 2 inverse terminal repeat sequences of AAV2 (SEQ ID 1).

2. Vectors

AAV2/1 pseudotype recombinant vectors were prepared in 293 cells, as already described (4), by cotransfecting 3 plasmids: pAAV2 (U7smOPT-SD23/BP22) encoding the genome rAAV2, pXX6 bearing the adenovirus helper functions and pAAV1p1TRCO2 which supplies the rep and cap genes of AAV1. The concentrations of vectors varied between 10¹² and 10¹³ vector genomes (vg) ml⁻¹.

3. Animals and Delivery Methods

All the animal procedures were carried out according to the protocol approved by the institution and under strict conditions of biological containment. A first group of mdx mice (8 weeks old) received injections of 50 μl PBS (phosphate buffered saline) containing 10¹² (vg) AAV (U7-SD23/BP22) into the tibialis anterior muscle of the right rear leg. The contralateral muscles were used as controls. A second group of mdx mice of the same age were subjected to an intra-arterial infusion of 2×10¹³ vectors via the femoral artery. The mice were sacrificed at given times, the muscles were frozen in isopentane cooled by liquid nitrogen and stored at −80° C.

4. Histology

Serial transverse sections (8 μm), made at 200 μm intervals along the length of the muscle, were examined for dystrophin (NCL-DYS2; murine monoclonal antibodies against the C-terminal domain; Novocastra) and the proteins associated with dystrophin (β-dystroglycan, α and β-sarcoglycan; Novocastra) by immunological detection, according to the manufacturer's instructions. The monoclonal antibodies were detected with biotinylated antibodies followed by avidin-FITC (M.O.M. Kit, Vector Laboratories). The sections prepared were analysed by laser scanning confocal microscopy (Leica). Intermediate tissues were collected for later analyses of proteins and RNA.

5. Analysis by Immunological Detection

The sections of the intermediate layers were collected and extracted with a lysis buffer containing 4% SDS, 125 mM Tris-HCl pH 6.4, 4 M urea, 10% β-mercaptoethanol, 10% glycerol, 0.001% bromophenol blue. After separation by centrifuging, the protein content was measured using the Bio-Rad Protein Assay test. The samples, adjusted to 40 μg of proteins, were loaded onto 6% polyacrylamide gels, subjected to electrophoresis and transferred onto nitrocellulose membranes which had been incubated with either NCL-DYS1 (murine monoclonal antibodies against the R8 repeated sequence of the spectrin-like rod domain of dystrophin; Novocastra) or NCL-DYS2, diluted to 1:100, followed by incubation with horseradish peroxidase conjugated secondary antibodies (1:1000) and analysis using the ECL Analysis System (Amersham).

6. RNA Analysis

The total RNA was isolated from a pool of intermediate sections using the reagent TRIzol (Life Technologies). To detect U7 and U7smOPT-SD23/BP22, reverse transcription was first of all carried out on the total RNA with Superscript II reverse transcriptase, in the presence of random hexamers (Invitrogen). Then, the cDNAs were amplified using Taq polymerase (Promega) with 5′-AAGTGTTACAGCTCTTTTAG-3′ (SEQ ID 18 located in the wild U7) or 5′-AAGGCCAAACCTCGGCTTAC-3′ (SEQ ID 19 located in U7smOPT-SD23/BP22) and 5′-AGGGGTTTTCCGACCGAAG-3′ (SEQ ID 20) for 30 cycles (94° C./30 s; 55° C./30 s; 72° C./30 s). The PCR products were analysed on 2% agarose gels. To detect the dystrophin mRNA, nested RT-PCR was carried out with 200 ng of total RNA. The first reaction occurred with the primers Ex20ext (SEQ ID 21; 5′CAGAATTCTGCCAATTGCTGAG-3′) and Ex26ext (SEQ ID 22; 5′-TTCTTCAGCTTGTGTCATCC-3′) in 30 cycles (94° C./30 s; 55° C./1 min; 72° C./2 min). Then 2 μl of the first reaction were amplified in 23 cycles with Ex20int (SEQ ID 23; 5′-CCCAGTCTACCACCCTATCAGAGC-3′) and Ex26int (SEQ ID 24; 5′-CCTGCCTTTAAGGCTTCCTT-3′). The PCR products were analysed on 2% agarose gels and the specific bands were purified for sequence analysis.

7. Physiology of the Muscle

The extensor digitorum longus muscles (EDL) of control or treated mice were dissected to assess their contractile/mechanical properties. The isolated muscles were connected on one side to an electromagnetic puller and on the other side to a force sensor, and were stimulated using electrodes placed parallel to the muscle. The tonic isometric contractions linked to a brief shock (125 Hz; 360 ms, separated by resting periods of 3 min) were studied at L₀ (the length at which the maximum isometric tonic force was observed). The isometric tension was calculated by dividing the force by the estimated cross-sectional area (CSA) of the muscle. Supposing that the muscles were cylindrical in shape and had a density of 1.06 mg.mm⁻³, the CSA corresponds to the wet weight of the muscle divided by the length of its fibres (5). Eccentric contractions induce muscle damage related to membrane rupture in a characteristic manner. They occur when a maximally contracted muscle is forcibly stretched, which leads to a loss of force. Here, the muscles were elongated by 10% of the length L₀ for which the muscle produced a maximum force at a speed of 1 fibre length per second. Five eccentric contractions were applied at 3 minute intervals. The accumulated decline in isometric force was quantified as previously described (5).

### II) Results

A U7 type RNA was modified in order to introduce antisense sequences into it which would interfere with the messenger RNA (mRNA) maturation process in the nucleus. The U7snRNA sequence was optimised to transport antisense sequences directed against introns 22 and 23 of the murine dystrophin gene. The sequences in intron 22 were chosen to compete with the fixation of U2snRNA at the BP (Branching Point) splice site (BP22; SEQ ID 3), and sequences in intron 23 corresponding to the fixation site of U1 at the donor site (SD23; SEQ ID 2) (FIG. 1). These sequences were used in a double target strategy, as recommended by Brun et al. (2).

The modified U7 gene, including both the promoter and the 3′ elements, was put into a construction based on AAV-2, which was introduced into the AAV-1 capsid to obtain high transfer efficiency for the transfer of genes into skeletal muscles. Adult mdx mice (8 weeks old) received a single dose of 10¹⁰ vector genomes by injection into the tibialis anterior muscle and were analysed at various times between 2 and 16 weeks. Molecular analysis of the skipping of exon 23 was done by nested RT-PCR on the total RNA prepared from the muscles injected. A shorter transcript, lacking exon 23, was detected. It represented 5 to 10% of the amplified material 2 weeks after injection and became the majority type after a month (FIGS. 2B and 2C). This slow accumulation of transcripts that had undergone exon skipping is not the result of the gradual expression of the transgene during the first weeks following the transfer of genes mediated by AAV, as the levels of the modified U7, measured at 2 and 4 weeks, were equivalent and were 5 times the level of the endogenous U7snRNA (FIG. 2A). This rather suggests limited availability of pre-mRNA and slow rotation of matured dystrophin mRNA in the muscle fibres.

In line with the generation of transcripts which had undergone exon skipping, the dystrophin protein was detected both by immunodetection in muscle extracts and by immunofluorescence on tissue sections (FIG. 3). The levels of dystrophin reflected those of the mRNA produced (0.5% to 30% of the normal level at 2 and 4 weeks respectively). Exon skipping produced immunoreactive protein species of the expected size, without evidence of the presence of multiple breakdown products. It should be noted that the difference in molecular weight between the wild and truncated protein cannot be resolved on the gel shown. Virtually all the fibres of the muscle which received the injection were positively detected from 4 weeks after the injection and the protein was typically located at the periphery of the fibres. Histological sections of treated muscles distinctly showed the disappearance of the dystrophic phenotype, with fibres that had regained their normal polygonal shape and the absence of inflammatory cells.

A group of mdx animals received the AAV-U7 vector by high-pressure intra-arterial perfusion in the lower limb. After a month this resulted in effective restoration of dystrophin in most fibres, in all the muscles of the perfused limb, including the TA and EDL muscles (FIG. 3F). In addition to dystrophin, the associated proteins including α-sarcoglycan, β-sarcoglycan and β-dystroglycan were detected at the periphery of the fibres of treated animals (FIG. 4). This confirmed that the mRNA product which undergoes exon skipping contains a C-terminal dystroglycan fixation domain which is essential for the anchoring function of dystrophin to the membrane.

The susceptibility to damage induced by exercise was assessed in treated animals by measuring the resistance to tonic contractions followed by forced elongation. For this experiment, the mice received a single dose of AAV-U7 into the EDL muscle and were analysed after 45 days. While the muscles of mdx animals were incapable of withstanding repeated elongations, the treated muscles expressing dystrophin restored in more than 70% of their fibres essentially performed in a way equivalent to normal muscles with a loss of force of 17% following 5 stimuli, compared with 15% for the wild type (FIG. 5A). Damage induced by the exercise was also evaluated by subjecting the mdx mice injected in the tibialis anterior muscle to an extensive course of movement, followed by an injection of Evans blue, a stain to which the cells are impermeable. Muscle lesions, revealed by the stain entering the fibres, were significant in the muscle treated (FIG. 5B) and absent in the contralateral, untreated muscle of the same animal (FIG. 5C).

### B.—IN VIVO STUDY IN THE DOG

The results obtained in the mouse could be transposed to a large animal, the GRMD dog. The GRMD dog has a point mutation in the splice acceptor site of intron 6 (AG transformed into GG; FIG. 6A), preventing the inclusion of exon 7 in the dystrophin mRNA (FIG. 6B). The mRNA thus formed (delta exon 7) has a reading frame shift linked to exons 6 and 8 being out of phase (FIGS. 6B and C).

Two U7 type RNAs were modified in order to introduce antisense sequences into them which would interfere with the splicing mechanism in the region of exons 6 and 8 (FIG. 7A), so that exons 6, 7 and 8 would not be taken into account in the final mRNA (multi-skipping; FIG. 7B). This mRNA, with a restored reading frame, enables a protein to be produced which is slightly truncated in the ABD domain (FIG. 7C), but theoretically perfectly functional.

The efficacy of AAV (U7-ex6) vectors integrating the antisense sequence SEQ ID 27 and AAV (U7-ex8) integrating the antisense sequence SEQ ID 28 was tested in vivo by local/regional injection in adult GRMD subjects. FIG. 8 shows the level of dystrophin obtained 2 months after a single intramuscular injection of a preparation of 500 μl containing a mixture of the two vectors (˜10¹¹ viral particles). This dystrophin was also detectable using western blot (FIG. 9). As in the mouse, the protein complex associated with dystrophin is also restored indirectly, suggesting that the quasi-dystrophin, induced by multiple exon skipping, is correctly functioning.

### C—IN VITRO STUDY IN MAN

We have developed an approach in which cell therapy and exon skipping can be combined in populations of cells capable of participating in muscle regeneration in vivo. To do this, we have developed lentiviral vectors carrying U7 cassettes to induce therapeutic skipping (murine, canine and human models).

As an example, we describe below the restoration of dystrophin in the cells of a DMD patient with deletion of exon 49-50.

Different Lenti (U7ex51 human) vectors were produced, one of them carrying the sequences previously described by De Angelis (1) (FIGS. 10A and B). Our results show that, under identical conditions of transduction, culture and analysis, the previously published target sequences (U7-ASDS) only function to a very limited extent and do not restore a dystrophin level compatible with rational therapy (FIG. 10C). On the other hand, the (U7-H51ab) vector, integrating the antisense sequences SEQ ID 5 and SEQ ID 6, has proved to be extremely efficient and almost completely modifies the dystrophin mRNAs which here are all cleared of exon 51 targeted by the vector (FIG. 10C).

Circulating AC133+ stem cells (20×10⁴) freshly obtained from a DMD patient (delta 49-50) were transduced by the Lent (U7-H51ab) vector integrating the antisense sequences SEQ ID 5 and SEQ ID 6, and rapidly reinjected into the tibialis anterior muscles of SCID-mdx mice, with the intention of proving the possibility of therapeutic exon skipping in vivo using cells from DMD patients.

Histological analysis, one and a half months later, conclusively demonstrated the restoration of human dystrophin (revealed using DYS3 antibodies not crossing with the murine dystrophin) in numerous, probably man/mouse chimeric, muscle fibres (FIG. 11).

### BIBLIOGRAPHY

1) De Angelis F G, Sthandier O, Berarducci B, Toso S, Galluzzi G, Ricci E, Cossu G, Bozzoni I, “Chimeric snRNA molecules carrying antisense sequences against the splice junctions of exon 51 of the dystrophin pre-mRNA induce exon skipping and restoration of a dystrophin synthesis in Delta 48-50 DMD cells”, PNAS U.S.A. 2002 Jul. 9; 9 (14):9456-61.
2) Brun C, Suter D, Pauli C, Dunant P, Lochmuller H, Burgunder J M, Schumperli D, Weis J, “U7 snRNAs induce correction of mutated dystrophin pre-mRNA by exon skipping”, Cell Mol Life Sci. 2003 March; 60 (3):557-66.
3) Gorman L, Suter D, Emerick V, Schumperli D, Kole R, “Stable alteration of pre-mRNA splicing patterns by modified U7 small nuclear RNAs”, PNAS U.S.A. 1998; 95:4929-34.
4) Snyder R O et al., “Efficient and stable adeno-associated virus-mediated transduction in the skeletal muscle of adult immunocompetent mice”, Hum Gene There 1997; 8: 1891-900.
5) Fougerousse F, Gonin P, Durand M, Richard I, Raymackers J M, “Force impairment in calpain 3-deficient mice is not correlated with mechanical disruption”, Muscle Nerve 2003; 27: 616-23.

1. An adeno-associated viral vector comprising:
a U7 type modified snRNA sequence;
the native U7 promoter;
at least one antisense sequence directed against at least one splice site of at least one exon, said at least one exon encoding a dispensable domain of dystrophin.

2. The vector according to claim 1, wherein said vector comprises a serotype 1 capsid.

3. The vector according to claim 2 wherein said vector is a 2/1 pseudotype.

4. The vector according to claim 1 wherein the at least one splice site is chosen from the group consisting of a 5′ donor site, a 3′ acceptor site, a BP (Branch Point) sequence and an exon-internal splicing enhancer (ESE) sequence.

5. The vector according to claim 1 comprising two antisense sequences.

6. The vector according to claim 5 wherein the antisense sequences are directed against the 5′ donor site and the BP sequence, respectively.

7. The vector according to claim 6 wherein the antisense sequences are SEQ ID NO.: 2 and SEQ ID NO.: 3 respectively.

8. The vector according to claim 7 comprising a nucleotide sequence of SEQ ID NO.1.

9. The vector according to claim 4, comprising an antisense sequence directed against an ESE sequence of exon 6 or exon 8 of the canine dystrophin gene.

10. The vector according to claim 1 wherein the at least one antisense sequence is directed against at least one splice site of at least one exon of the human dystrophin gene.

11. The vector according to claim 10 wherein the at least one exon is exon 51.

12. The vector according to claim 11 wherein the at least one antisense sequence comprises a sequence selected from the group comprising SEQ ID NO.: 5, SEQ ID NO.: 6, SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, SEQ ID NO.: 25 and SEQ ID NO.: 26.

13. The vector according to claim 12 comprising the sequences SEQ ID NO.: 5 and SEQ ID NO.: 6, or SEQ ID NO.: 7 and SEQ ID NO.: 8, or SEQ ID NO.: 9 and SEQ ID NO.: 10, or SEQ ID NO.: 25 and SEQ ID NO.: 26.

14. The vector according to claim 13 comprising SEQ ID NO.: 4.

15. The vector according to claim 1 wherein the at least one antisense sequence is directed against splice sites of at least two distinct exons.

16. A lentiviral vector comprising:
a U7 type modified snRNA sequence;
the native U7 promoter;
at least one antisense sequence chosen from the group consisting of SEQ ID NO.: 2, SEQ ID NO.: 3, SEQ ID NO.: 27, SEQ ID NO.: 28, SEQ ID NO.: 5, SEQ ID NO.: 6, SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, SEQ ID NO.: 25, SEQ ID NO.: 26.

17. An isolated cell transfected by a vector according to claim 1.

18. The isolated cell according to claim 17 wherein said cell is a muscle cell.

19. The isolated cell according to claim 18 wherein said muscle cell is a myoblast or a cell capable of muscle differentiation.

20. Isolated muscle tissue comprising cells transfected by a vector according to claim 1.

21. A non-human organism comprising cells transfected by a vector according to claim 1.

22. A pharmaceutical composition comprising a vector according to claim 1.

23. (canceled)

24. An isolated cell transfected by a vector according to claim 16.

25. The isolated cell according to claim 24 wherein said cell is a muscle cell.

26. The isolated cell according to claim 25 wherein said muscle cell is a myoblast or a cell capable of muscle differentiation.

27. Isolated muscle tissue comprising a cell transfected by a vector according to claim 16.

28. A non-human organism comprising cells transfected by a vector according to claim 16.

29. A pharmaceutical composition comprising a vector according to claim 16.

30. (canceled)

31. A pharmaceutical composition comprising a cell according to claim 17.

32. (canceled)

33. The vector according to claim 9, wherein the ESE sequence comprises SEQ ID NO.: 27 or SEQ ID NO.: 28.

34. A method for restoring functional dystrophin to a tissue, the method comprising:
contacting said tissue with an adeno-associated viral vector comprising:
a U7 type modified snRNA sequence;
the native U7 promoter; and
at least one antisense sequence directed against at least one splice site of at least one exon, said at least one exon encoding a dispensable domain of dystrophin under conditions where cells of said tissue are transfected with the vector.

35. The method of claim 34 wherein said individual suffers from Duchenne muscular dystrophy.

36. A method for restoring functional dystrophin to a tissue, the method comprising:
(a) contacting a cell with an adeno-associated viral vector comprising:
a U7 type modified snRNA sequence;
the native U7 promoter; and
at least one antisense sequence directed against at least one splice site of at least one exon, said at least one exon encoding a dispensable domain of dystrophin under conditions where said cell is transfected with the vector;
(b) injecting said cell into muscle tissue of an individual in need of dystrophin restoration.

37. The method of claim 36 wherein said individual suffers from Duchenne muscular dystrophy.

38. A lentiviral vector comprising: a U7 type modified snRNA sequence; the native U7 promoter; and at least one antisense sequence directed against at least one splice site of one or more exons of the human dystrophin gene.


SEQUENCE LISTING (28 sequences)

SEQ ID NO: 1 - DNA, 960 bp, Artificial Sequence
  misc_feature 1..960: U7-SD23/BP22 cassette
ttgggaagggcgatcggtgcgggcctcttcgctattacgccagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagataacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggccaaacctcggcttacctaaatagaagttcatttacactaacaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtggctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcgg

SEQ ID NO: 2 - DNA, 20 bp, Artificial Sequence
  misc_feature 1..20: splice donor site (SD23)
ggccaaacctcggcttacct

SEQ ID NO: 3 - DNA, 24 bp, Artificial Sequence
  misc_feature 1..24: branch point sequence(BP22)
aaatagaagttcatttacactaac

SEQ ID NO: 4 - DNA, 468 bp, artificial sequence
  misc_feature 1..468: U7-h51AON2/h51AON1 cassette
gggtctagataacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaacctctgtgattttataacttgattcaaggaagatggcatttctaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtggctagcttt

SEQ ID NO: 5 - DNA, 23 bp, artificial sequence
  misc_feature 1..23: h51AON2
cctctgtgattttataacttgat

SEQ ID NO: 6 - DNA, 20 bp, artificial sequence
  misc_feature 1..20: h51AON1
tcaaggaagatggcatttct

SEQ ID NO: 7 - DNA, 24 bp, artificial sequence
  misc_feature 1..24: DA5′
tttttctcataccttctgcttcat

SEQ ID NO: 8 - DNA, 28 bp, artificial sequence
  misc_feature 1..28: DA3′
gtctgagtaggagctaaaatattttggg

SEQ ID NO: 9 - DNA, 20 bp, artificial sequence
  misc_feature 1..20: G5′
tatcattttttctcatacct

SEQ ID NO: 10 - DNA, 22 bp, artificial sequence
  misc_feature 1..22: GBP
tttgggtttttgcaaagaagga

SEQ ID NO: 11 - DNA, 26 bp, artificial sequence
  misc_feature 1..26: ESE4
tgtcaccagagtaacagtctgagtag

SEQ ID NO: 12 - DNA, 25 bp, artificial sequence
  misc_feature 1..25: ESE16
accacaggttgtgtcaccagagtaa

SEQ ID NO: 13 - DNA, 25 bp, artificial sequence
  misc_feature 1..25: ESE28
gtttccttagtaaccacaggttgtg

SEQ ID NO: 14 - DNA, 19 bp, artificial sequence
  misc_feature 1..19: U7-1 murin
taacaacataggagctgtg

SEQ ID NO: 15 - DNA, 20 bp, artificial sequence
  misc_feature 1..20: U7-2 murin
cagatacgcgtttcctagga

SEQ ID NO: 16 - DNA, 11 bp, artificial sequence
  misc_feature 1..11: Sm
aatttgtctag

SEQ ID NO: 17 - DNA, 11 bp, artificial sequence
  misc_feature 1..11: SmOPT
aatttttggag

SEQ ID NO: 18 - DNA, 20 bp, artificial sequence
  misc_feature 1..20: primer U7
aagtgttacagctcttttag

SEQ ID NO: 19 - DNA, 20 bp, artificial sequence
  misc_feature 1..20: primer U7smOPT-SD23/BP22
aaggccaaacctcggcttac

SEQ ID NO: 20 - DNA, 19 bp, artificial sequence
  misc_feature 1..19: primer commun U7
aggggttttccgaccgaag

SEQ ID NO: 21 - DNA, 22 bp, artificial sequence
  misc_feature 1..22: Ex20ext
cagaattctgccaattgctgag

SEQ ID NO: 22 - DNA, 20 bp, artificial sequence
  misc_feature 1..20: Ex26ext
ttcttcagcttgtgtcatcc

SEQ ID NO: 23 - DNA, 24 bp, artificial sequence
  misc_feature 1..24: Ex20int
cccagtctaccaccctatcagagc

SEQ ID NO: 24 - DNA, 20 bp, artificial sequence
  misc_feature 1..20: Ex26int
cctgcctttaaggcttcctt

SEQ ID NO: 25 - DNA, 45 bp, artificial sequence
  misc_feature 1..45: ex51AONlong1
gcaggtacctccaacatcaaggaagatggcatttctagtttggag

SEQ ID NO: 26 - DNA, 45 bp, artificial sequence
  misc_feature 1..45: ex51AONlong2
ccctctgtgattttataacttgatcaagcagagaaagccagtcgg

SEQ ID NO: 27 - DNA, 41 bp, artificial sequence
  misc_feature 1..41: C6ESE2
acattaacctgtggataattacgagttgatttcggacccag

SEQ ID NO: 28 - DNA, 39 bp, artificial sequence
  misc_feature 1..39: C8ESE1
acttgttgaggcaaaacttggaagagtgatgtgatatac